(19) United States
(12) Patent Application Publication
(10) Pub. No.: US 2010/0226906 A1
(43) Pub. Date: Sep. 9, 2010

(54) Recombinant poly-glutamic acid depolymerases

(76) Inventors: Arthur M. Friedlander, Montgomery Village, MD (US); Angelo Scorpio, Frederick, MD (US); Donald J. Chabot, Frederick, MD (US)

Correspondence Address: U.S. Army Medical Research and Materiel Commnad;Attn: MCMR-JA (Ms. Elizabeth Arwine), 504 Scott Street, Fort Detrick, MD, 21702-5012, US

(21) Appl. No.: 11/523,176

(22) Filed: Sep. 19, 2006

Related U.S. Application Data
(60) Provisional application No. 60/726,758, filed on Sep. 20, 2005.

(51) Int. Cl. A61K 38/45; C12N 9/10; C12N 15/63; C12N 9/48; A61K 38/48; C12N 1/19; C12N 1/21; C12N 5/10; A61P 31/04

(52) U.S. Cl. 424/94.5; 435/193; 435/320.1; 435/110; 435/212; 424/94.63; 435/254.2; 435/254.21; 435/254.23; 435/252.33; 435/252.31; 435/252.35; 435/252.3; 435/358; 435/369; 435/325; 435/367; 435/366; 435/348; 435/364

(57) ABSTRACT

In this application is described isolated, recombinant CapD and recombinant PghP for use in digesting capsule comprising polyglutamate polymers and for treatment of infections caused by bacilli having a polyglutamate capsule, such as anthrax.

The benefit of priority under 35 U.S.C., Section 119(e) is hereby claimed from U.S. Provisional Ser. No. 60/726,758 filed on Sep. 20, 2005, still pending.

### INTRODUCTION

Bacillus anthracis, the causative agent of anthrax, produces a plasmid encoded poly-γ-D-glutamic acid capsule (Green et al., 1985, Infect. Immun. 49, 291-297) that shields the bacterium from phagocytic cells (Chabot et al., 2004, Vaccine 23, 43-47; Ezzell and Welkos, 1999, J. Appl. Microbiol. 87, 250). The antiphagocytic property of the capsule is the primary mechanism of immune cell evasion utilized by B. anthracis and is essential for virulence (Drysdale et al., 2005, EMBO J. 24, 221-227; Keppie et al, 1063, Br. J. Exp. Pathol. 44, 446-453; Makino et al., 1989, J. Bacteriol. 171, 722-730). In addition to the capsule, B. anthracis also produces an AB type toxin consisting of protective antigen (PA), lethal factor (LF) and edema factor (EF). LF has been characterized as a zinc-dependent metalloprotease that cleaves and inactivates components of the MAPK signal transduction pathway (Duesbery et al., 1998, Science 280, 734-737), while EF is an adenylate cyclase that increases intracellular concentrations of cAMP (Leppla, S. H.1982, Proc. Natl. Acad. Sci. USA 79, 3162-3166).

Neutrophils are the primary component of innate immunity and are vital for elimination of bacteria from the blood stream and from sites of infection. Following phagocytosis, neutrophils release intracellular granules in the phagolysosome which contain reactive oxygen intermediates and antibacterial enzymes that rapidly kill ingested bacteria. While neutrophils are reported to have a minimal impact on the initial stages of anthrax spore infection in mice (Cote et al, 2006, Infect. Immun. 74, 469-480), it has been demonstrated that human neutrophils are highly efficient in killing the vegetative form of B. anthracis and can reduce viability by up to 3 logs (Mayer-Scholl et al., 2005, PLOS Pathog 1, 179-186; Scorpio et al., 2005, presented at the Bacillus anthracis, B. cereus, and B. thuringiensis International Conference, Santa Fe, N. Mex., USA, Sep. 25-29, 2005). B. anthracis has evolved mechanisms to subvert the bactericidal activity of neutrophils that include synthesis of the tripartite toxin and the polyglutamate capsule. The anthrax toxin complex is known to inhibit neutrophil function and actin assembly (Abalakin et al., 1990, Zh Mikrobiol. Epidemiol. Immunobiol. 62-67; Alexeyev et al., 1994, Infection 22, 281-282; Crawford et al., 1006 J. Immunol. 176, 7557-7565; During et al., 2005, J. Infect. Dis. 192, 837-845; O'Brien et al., 1985, Infect. Immun. 47, 306-310; Wright and Mandell, 1986, J. Exp. Med. 164, 1700-1709) but in contrast with its deleterious effect on macrophage survival (Friedlander et al., 1993, Infect. Immun. 61, 245-252), does not affect human neutrophil viability (Crawford et al., 2006, J. Immunol. 176, 7557-7565). Capsule has long been proposed to be strongly antiphagocytic (Keppie et al., 1963, Br. J. Exp. Pathol. 44, 446-453; Makino et al., 1989, J. Bacteriol. 171, 722-730), although the exact mechanism remains to be established, and probably allows virtually unimpeded growth of anthrax bacilli in the host. As such, the toxins may play a less significant role than capsule in promoting dissemination of anthrax bacilli and progression to septicemia. Strategies to negate the antiphagocytic effect of capsule may thus lead to methods that facilitate clearance of circulating bacilli from the blood stream and eventual resolution of infection. Recent reports have demonstrated that anti-capsule antibodies can opsonize B. anthracis and confer protection in mouse models of anthrax infection (Chabot et al., 2004, Vaccine 23, 43-47; Kozel et al., 2004, Proc. Natl. Acad. Sci. USA 101, 5042-5047; Schneerson et al., 2003, Proc. Natl. Acad. Sci. USA 100, 8945-8950; Wang et al., 2004, FEMS Immunol. Med. Microbiol. 40, 231-237) and suggest that methods to target B. anthracis to phagocytic cells may be viable strategies to combat infection. Additionally, adhesion experiments with unencapsulated bacilli showed that they are highly susceptible to leukocyte phagocytosis (Keppie et al., 1953, Br. J. Exp. Pathol. 34, 486-496; Makino et al., 1989, J. Bacteriol. 171, 722-730) and suggest that removal of the capsule from the bacilli surface may potentially lead to similar levels of phagocytosis.

The strategy of enzymatically removing bacterial capsule from the surface of microorganisms as an approach to treat infections dates back to 1931 with the work of Avery and Dubos (Avery and Dubos, 1931, J. Exp. Med. 54, 73-89). It was demonstrated in these studies that injection of an enzyme capable of degrading the pneumococcal capsular polysaccharide could protect mice from pneumococcal infection, presumably by targeting the bacteria to phagocytic cells. Although the advent of antibiotics curtailed this approach to therapy, the emergence of antibiotic resistant bacterial pathogens may signal a renewed interest in such treatments. The recent work of Mushtaq et al. demonstrated that a capsule degrading endosialidase could be used to treat E. coli infections in mice, again by targeting the bacteria for phagocytic killing (Mushtaq et al., 2004, Antimicrob. Agents Chemother. 48, 160-165; Mushtaq et al., 2005, J. Antimicrob. Chemother. 24, 160-165).

CapD is a poly-γ-D-glutamic acid specific protease that is autocatalytic and forms a heterodimer consisting of 35 kDa and 15 kDa polypeptides. It is thought to contribute to the virulence of B. anthracis by releasing low molecular weight capsule from the surface of the bacilli (Makino et al., 1989, supra) and has been demonstrated to be involved in anchoring the capsule to the peptidoglycan layer (Candela and Fouet 2005, Mol. Microbiol. 57, 717-726). When added exogenously to encapsulated bacilli, however, the enzyme efficiently degrades the capsule, essentially removing it from the surface of the bacilli (Scorpio et al., 2005, supra). We have developed a method to remove the capsule from the surface of the bacillus with recombinant CapD enzyme and thereby render the bacteria susceptible to neutrophil killing.

Poly-γ-glutamate hydrolase (PghP) is a 25 kDa enzyme encoded by the bacteriophage φNIT1 that specifically cleaves D- and L-polyglutamic acid, a component of the capsule produced by several strains of Bacillus subtilis. The phage is a common contaminant in B. subtilis natto cultures and causes markedly reduced viscosity of the cultures from capsule hydrolysis, a significant problem in natto factories. The enzyme has been shown to be an important factor in the ability of the phage to infect B. subtilis strains that produce poly-glutamic acid capsule (Kimura and Itoh, 2003, Appl. Environ. Microbiol. 69, 249-247). In vitro analysis has shown that PghP rapidly cleaves high molecular weight B. subtilis capsule (5×10⁶ Da) to trimers within 45 minutes (Kimura and Itoh, 2003, supra).

In this application, we show that recombinant CapD and PghP degrade high molecular weight capsule and opsonize encapsulated B. anthracis Ames bacilli, targeting the organisms for phagocytocis and killing by human neutrophils and mouse macrophages. Additionally, encapsulated bacilli treated with the enzymes adhered to mouse macrophages at a higher rate that untreated bacilli. These data suggest that recombinant CapD and PghP enable phagocytes to ingest and kill bacilli by removing the capsule from the surface of the organism. Furthermore, we report here that recombinant CapD protected mice from anthrax infection by removing the capsule and allowing phagocytic killing of bacilli in vivo. When co-injected with a bacillus challenge, CapD conferred 100% protection from challenge with the fully virulent Ames strain of B. anthracis. Additionally, when CapD was administered 30 hours after infection with spores of the encapsulated, non-toxigenic strain, Δames, significant protection was observed compared with a PBS or irrelevant protein control. Prei-ncubation of human neutrophils with either lethal or edema toxin did not inhibit the bactericidal activity of neutrophils suggesting that targeting anthrax bacilli to neutrophils is a sound strategy to treat existing anthrax infection.

### SUMMARY OF THE INVENTION

The present invention provides for recombinant CapD and PghP enzymes, efficient in degrading high molecular weight poly-glutamic acid. The poly-glutamic acid degrading activity when aimed at capsule of bacilli, promotes opsonization of encapsulated B. anthracis bacilli and targets the organisms for phagocytosis and killing by human neutrophils and macrophages.

Therefore, it is an object of the present invention to provide recombinant CapD for use in degrading a poly-γ-D-glutamic acid polymer, for use in a diagnostic assay, and as a therapeutic for infections by organisms producing a poly-γ-D-glutamic acid polymer.

It is another object of the present invention to provide a method for degrading poly-γ-D-glutamic acid by providing CapD in an amount sufficient to degrade said polymer.

It is yet another object of the present invention to provide recombinant PghP for use in degrading a poly-γ-DL-glutamic acid polymer, for use in a diagnostic assay, and as a therapeutic for infections by organisms producing a poly-γ-DL-glutamic acid polymer.

It is still another object of the present invention to provide a method for degrading poly-γ-DL-glutamic acid polymer by providing PghP in an amount sufficient to degrade said polymer.

It is another object of the invention to provide a composition comprising recombinant CapD and/or PghP.

It is yet another object of the present invention to provide novel vector constructs for recombinantly expressing CapD and/or PghP, as well as host cells transformed with said vector.

It is also an object of the present invention to provide a method for producing and purifying recombinant CapD and/or PghP protein comprising:

growing a host cell containing a vector expressing CapD and/or PghP proteins in a suitable culture medium,

causing expression of said vector sequence as defined above under suitable conditions for production of soluble protein and,

lysing said transformed host cells and recovering said CapD and/or PghP protein.

It is also an object of the present invention to provide a therapy for a variety of illnesses caused by organisms producing a capsule comprising poly-γ-D-glutamic acid polymer, comprising administering a composition comprising CapD.

In yet another object of the present invention is provided a therapeutic composition for anthrax infection comprising CapD. The therapy may also include conventional antibiotics.

It is another object of the present invention to provide a therapy for a variety of illnesses caused by organisms producing a capsule comprising a poly-γ-DL-glutamic acid polymer, said therapy comprising administering a composition comprising PghP.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### BRIEF DESCRIPTION OF THE DRAWINGS

FIG. 1: CapD 43-528 and CapD 28-528 degrade B. antracis capsule. Purified capsule was incubated with PBS (A), CapD 43-528 (B) or CapD 28-528 (C) and examined by agarose gel electrophoresis.

FIG. 2: CapD treatment results in increased survival (P=0.035) following infection with 1000 CFU B. anthracis ΔAmes bacilli. Swiss Webster mice were challenged with ΔAmes bacilli treated with either CapD or PBS as described in the Materials and Methods and monitored for mortality.

FIG. 3: CapD treatment results in an increased survival curve (P=0.0003) following infection with fully virulent B. anthracis Ames bacilli. Swiss Webster mice were challenged with 4,000 CFU Ames bacilli treated with either CapD or heat inactivated CapD as described in the Materials and Methods and monitored for mortality.

FIG. 4: CapD treatment results in increased survival (P<0.0001) following infection with fully virulent B. anthracis Ames bacilli. Swiss Webster mice were challenged with 500 CFU Ames bacilli treated with either CapD or heat inactivated CapD as described in the Materials and Methods and monitored for mortality. A control group was included that did not receive 1 ml starch solution 6 h prior to challenge.

FIG. 5: CapD administration increases survival (P=0.005) following challenge with B. anthracis ΔAmes spores. Swiss Webster mice were injected i.p. and i.v. with CapD, concurrently with spore challenge and 30 h following spore challenge and monitored for mortality.

FIG. 6: CapD administration increases survival (P=0.035) compared with an irrelevant protein when given 30 h after challenge with B. anthracis ΔAmes spores. Swiss Webster mice were injected i.p. and i.v. with CapD or recombinant BA3927 protein 30 h after spore challenge and monitored for mortality.

FIG. 7: Heat inactivated CapD at high concentration induces neutrophil killing of B. anthracis bacilli.

FIGS. 8A and 8B: CapD treatment does not significantly effect survival when administered 30 h after challenge with B. anthracis Ames spores. Swiss Webster mice were injected i.p. and i.v. with CapD or heat inactivated CapD 30 h after spore challenge and monitored for mortality. Two identical experiments (A and B) were performed at different times.

### DETAILED DESCRIPTION

In the description that follows, a number of terms used in recombinant DNA, microbiology and immunology are extensively utilized. In order to provide a clear and consistent understanding of the specification and claims, including the scope to be given such terms, the following definitions are provided.

The term ‘purified’ as applied to proteins herein refers to a composition wherein the desired protein comprises at least 35% of the total protein component in the composition. The desired protein preferably comprises at least 40%, more preferably at least about 50%, more preferably at least about 60%, still more preferably at least about 70%, even more preferably at least about 80%, even more preferably at least about 90%, and most preferably at least about 95% of the total protein component. The composition may contain other compounds such as carbohydrates, salts, lipids, solvents, and the like, without affecting the determination of the percentage purity as used herein.

The term ‘essentially purified proteins’ refers to proteins purified such that they can be used for in vitro diagnostic methods and as a prophylactic compound. These proteins are substantially free from cellular proteins, vector-derived proteins or other components. The proteins of the present invention are purified to homogeneity, at least 80% pure, preferably, 90%, more preferably 95%, more preferably 97%, more preferably 98%, more preferably 99%, even more preferably 99.5%.

The term ‘recombinantly expressed’ used within the context of the present invention refers to the fact that the proteins of the present invention are produced by recombinant expression methods be it in prokaryotes, or lower or higher eukaryotes as discussed in detail below.

The term ‘lower eukaryote’ refers to host cells such as yeast, fungi and the like. Lower eukaryotes are generally (but not necessarily) unicellular. Preferred lower eukaryotes are yeasts, particularly species within Saccharomyces, Schizosaccharomyces, Kluveromyces, Pichia (e.g. Pichia pastoris), Hansenula (e.g. Hansenula polymorpha, Yarowia, Schwaniomyces, Schizosaccharomyces, Zygosaccharomyces and the like. Saccharomyces cerevisiae, S. carlsberoensis and K. lactis are the most commonly used yeast hosts, and are convenient fungal hosts.

The term ‘prokaryotes’ refers to hosts such as E. coli, Lactobacillus, Lactococcus, Salmonella, Streptococcus, Bacillus subtilis or Streptomyces. Also these hosts are contemplated within the present invention.

The term ‘higher eukaryote’ refers to host cells derived from higher animals, such as mammals, reptiles, insects, and the like. Presently preferred higher eukaryote host cells are derived from Chinese hamster (e.g. CHO), monkey (e.g. COS and Vero cells), baby hamster kidney (BHK), pig kidney (PK15), rabbit kidney 13 cells (RK13), the human osteosarcoma cell line 143 B, the human cell line HeLa and human hepatoma cell lines like Hep G2, and insect cell lines (e.g. Spodoptera frugiperda). The host cells may be provided in suspension or flask cultures, tissue cultures, organ cultures and the like. Alternatively the host cells may also be transgenic animals.

The term ‘polypeptide’ refers to a polymer of amino acids and does not refer to a specific length of the product; thus, peptides, oligopeptides, and proteins are included within the definition of polypeptide. This term also does not refer to or exclude post-expression modifications of the polypeptide, for example, glycosylations, acetylations, phosphorylations and the like. Included within the definition are, for example, polypeptides containing one or more analogues of an amino acid (including, for example, unnatural amino acids, PNA, etc.), polypeptides with substituted linkages, as well as other modifications known in the art, both naturally occurring and non-naturally occurring.

The term ‘recombinant polynucleotide or nucleic acid’ intends a polynucleotide or nucleic acid of genomic, cDNA, semi-synthetic, or synthetic origin which, by virtue of its origin or manipulation: (1) is not associated with all or a portion of a polynucleotide with which it is associated in nature, (2) is linked to a polynucleotide other than that to which it is linked in nature, or (3) does not occur in nature.

The term ‘recombinant host cells’, ‘host cells’, ‘cells’, ‘cell lines’, ‘cell cultures’, and other such terms denoting microorganisms or higher eukaryotic cell lines cultured as unicellular entities refer to cells which can be or have been, used as recipients for a recombinant vector or other transfer polynucleotide, and include the progeny of the original cell which has been transfected. It is understood that the progeny of a single parental cell may not necessarily be completely identical in morphology or in genomic or total DNA complement as the original parent, due to natural, accidental, or deliberate mutation.

The term ‘replicon’ is any genetic element, e.g., a plasmid, a chromosome, a virus, a cosmid, etc., that behaves as an autonomous unit of polynucleotide replication within a cell; i.e., capable of replication under its own control.

The term ‘vector’ is a replicon further comprising sequences providing replication and/or expression of a desired open reading frame.

The term ‘control sequence’ refers to polynucleotide sequences which are necessary to effect the expression of coding sequences to which they are ligated. The nature of such control sequences differs depending upon the host organism; in prokaryotes, such control sequences generally include promoter, ribosomal binding site, and terminators; in eukaryotes, generally, such control sequences include promoters, terminators and, in some instances, enhancers. The term ‘control sequences’ is intended to include, at a minimum, all components whose presence is necessary for expression, and may also include additional components whose presence is advantageous, for example, leader sequences which govern secretion.

The term ‘promoter’ is a nucleotide sequence which is comprised of consensus sequences which allow the binding of RNA polymerase to the DNA template in a manner such that mRNA production initiates at the normal transcription initiation site for the adjacent structural gene.

The expression ‘operably linked’ refers to a juxtaposition wherein the components so described are in a relationship permitting them to function in their intended manner. A control sequence ‘operably linked’ to a coding sequence is ligated in such a way that expression of the coding sequence is achieved under conditions compatible with the control sequences.

An ‘open reading frame’ (ORF) is a region of a polynucleotide sequence which encodes a polypeptide and does not contain stop codons; this region may represent a portion of a coding sequence or a total coding sequence.

A ‘coding sequence’ is a polynucleotide sequence which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include but is not limited to mRNA, DNA (including cDNA), and recombinant polynucleotide sequences.

The term ‘therapeutic’ refers to a composition capable of treating an infection.

The term ‘effective amount’ for a therapeutic or prophylactic treatment refers to an amount of epitope-bearing polypeptide sufficient to facilitate clearance of polyglutamate capsule-producing bacteria from the individual to which it is administered. Preferably, the effective amount is sufficient to effect treatment, as defined above. The exact amount necessary will vary according to the application. For therapeutic applications, for example, the effective amount may vary depending on the species, age, and general condition of the individual, the severity of the condition being treated, the particular polypeptide selected and its mode of administration, etc. It is also believed that effective amounts will be found within a relatively large, non-critical range. An appropriate effective amount can be readily determined using only routine experimentation. Preferred ranges of CapD for prophylaxis are about 0.01 to 100,000 ug/dose, more preferably about 0.1 to 10,000 ug/dose, most preferably about 10-500 ug/dose. The minimal concentration of enzyme effective in promoting killing of B. anthracis bacilli by neutrophil is 1 ug/ml. Thus, an amount of at least 5000 ug, given intravenously would be needed as a dose to an individual with 5 liters of blood. Several doses may be needed per individual in order to achieve resolution of infection.

Poly-γ-D-glutamic acid is a high molecular weight polypeptide comprised of D-glutamate connected by a gamma linkage.

Poly-γ-DL-glutamic acid (PGA) is an anionic, extracellular polymer, in which the α-amino and the γ-carboxy groups of D- or L-glutamic acid are linked by isopeptide bonds. PGA is produced primarily by Bacillus strains but also by other strains of bacteria, archaebacteria, and some eukaryotes (Oppermann-Sanio and Steinbuchel, 2002, Naturwissenschaften 89, 11-22).

CapD, a capsule degrading enzyme encoded by the B. anthracis capD gene, proteolytically cleaves poly-γ-D-glutamic acid to a lower molecular weight form. Homologs of CapD can be found in other bacillus species such as B. subtillis and B. licheniformis.

Polyglutamate hydrolase, PghP, is a 25 kDa enzyme encoded by the bacteriophage, φNIT1 that specifically cleaves D- and L-polyglutamic acid, a component of the capsule produced by several strains of B. subtilis.

The present invention contemplates recombinant CapD enzyme or PghP enzyme and a method for isolating or purifying these recombinant proteins, characterized in that the recombinantly expressed protein retains its enzymatic ability, i.e. for CapD ability to degrade poly-γ-D-glutamic acid polymers, and for PghP, the ability to degrade poly-γ-DL-glutamic acid polymers.

The recombinant CapD protein of the present invention spans from amino acid 28-528 (Genbank™ Accession No. NC007323; gene ID No. 2820407 of the published sequence which represents the complete protein minus the signal sequence. The term CapD refers to a polypeptide or an analogue thereof (e.g. mimotopes) comprising an amino acid sequence (and/or amino acid analogues) defining at least one CapD epitope including the enzymatically active site. CapD is an approximately 50 kDa autocatalytic protein that self cleaves into 35 kDa and 15 kDa subunits. The subunits associate to form an active species that can degrade poly-γ-D-glutamate.

The CapD antigen used in the present invention is preferably a full-length protein as described above, or a substantially full-length version, i.e. containing functional fragments thereof (e.g. fragments which are not missing sequence essential to the formation or retention of enzyme activity) for example, spanning amino acid 30-528, or amino acids 43-528, to name a few. Furthermore, the CapD antigen of the present invention can also include other sequences that do not block or prevent the enzymatic activity of interest. The presence or absence of enzymatic activity can be readily determined through screening as described in the Examples below and comparing its activity to that of a denatured version of the antigen (if any).

The CapD antigen of the present invention can be made by any recombinant method that provides the enzyme of interest. For example, recombinant expression in E. coli is a preferred method to provide antigens. Proteins secreted from mammalian cells may contain modifications including galactose or sialic acids which may be undesirable for certain diagnostic or vaccine applications. However, it may also be possible and sufficient for certain applications, as it is known for proteins, to express the antigen in other recombinant hosts such as baculovirus and yeast or higher eukaryotes.

The proteins according to the present invention may be secreted or expressed within compartments of the cell. Preferably, however, the proteins of the present invention are expressed within the cell and are released upon lysing the cells.

It is also understood that the isolates used in the examples section of the present invention were not intended to limit the scope of the invention and that an enzyme equivalent to CapD from B. anthracis, i.e. which degrades poly-γ-D-glutamic acid, from another organism can be used to produce a recombinant enzyme using the methods described in the present application. Other species of bacteria such as B. circulans, B. licheniformis and S. epidermidis also produce enzymes (gamma-glutamyl transferases or ggt's) similar to capD that degrade poly-glutamic acid polymer.

The CapD of the present invention is expressed as part of a recombinant vector. The present invention relates more particularly to a polynucleotide sequence (SEQ ID NO:1), the capD gene encoding amino acids 28-528 of B. anthracis CapD (SEQ ID NO:2). The capD gene encoding amino acids 28-528 was cloned into pTYB12 and pET15b plasmids and expressed using systems which allow over-expression of a target protein as a fusion to a self-cleavable affinity tag or nickel binding hexa-histidine tag. Examples of other plasmids in which CapD can be expressed include, but are not limited to, pMAL (New England Biolabs) and pGEX (Promega Corp.). CapD was also expressed in pMAL as a fusion protein yielding functional enzyme but with higher solubility than a non-fusion form of CapD.

The open reading frame of the PghP gene, Genbank™ accession no. BAC65290 was amplified from phiNIT1 DNA and cloned into the pET15b expression vector. The enzyme can also be made by infecting B. subtilis natto (a capsule producing strain) with the phage PhiNIT1 to lyse the bacterial cells, and isolating the enzyme from the supernatant.

The present invention also contemplates host cells transformed with a recombinant vector as defined above. Any prokaryotic host can be used. In a preferred embodiment, E. coli strain is employed. The above plasmids may be transformed into this strain or other strains of E. coli. Other host cells such as insect cells can be used depending on the vector chosen and taking into account that other cells may result in lower levels of expression.

Eukaryotic hosts include lower and higher eukaryotic hosts as described in the definitions section. Lower eukaryotic hosts include yeast cells well known in the art. Higher eukaryotic hosts mainly include mammalian cell lines known in the art and include many immortalized cell lines available from the ATCC, inluding HeLa cells, Chinese hamster ovary (CHO) cells, Baby hamster kidney (BHK) cells, PK15, RK13 and a number of other cell lines. Methods for introducing vectors into cells are known in the art. Please see e.g., Maniatis, Fitsch and Sambrook, Molecular Cloning; A Laboratory Manual (1982) or DNA Cloning, Volumes I and II (D. N. Glover ed. 1985) for general cloning methods. Host cells provided by this invention include E. coli containing pTYBl2capD, pMALcapD, pET15 bpghp, or a plasmid encoding capD or pghp in other suitable expression systems. A preferred method for isolating or purifying CapD or PghP as defined above is further characterized as comprising:

(i) growing a host cell as defined above transformed with a recombinant vector encoding CapD or PghP protein in a suitable culture medium,

(ii) causing expression of said vector sequence as defined above under suitable conditions for production of a soluble protein,

(iii) lysing said transformed host cells and recovering said CapD or PghP protein.

At this point the recombinant protein is about poly-γ-D-glutamic 90% of total protein. Endotoxin can be removed from CapD preparations by column chromatography as described below in Materials and Methods or any other method known in the art.

The present invention more particularly relates to a composition comprising at least one of the above-specified recombinant enzymes as defined above, for use as a therapeutic composition, against infection with an organism having all or a portion of its capsule comprising a poly-γ-D-glutamic acid polymer or a poly-γ-DL-glutamic acid polymer. Examples of organisms having a capsule comprising a poly-γ-D-glutamic acid polymer include B. anthracis Examples of organisms having a capsule comprising a poly-γ-DL-glutamic acid polymer include B. subtilis, licheniformis, and Staphylococcus epidemidis.

Treatment of individuals having an infection comprises administering a therapeutic composition in a sufficient amount, possibly accompanied by pharmaceutically acceptable carrier, or other drugs known to promote clearing of the infections, e.g. antibiotics, in order to produce a reduction in symptoms of the infection. In general, this will comprise administering a therapeutically or prophylactically effective amount of one or both CapD or PghP of the present invention to a susceptible subject or one exhibiting infection symptoms. The proteins of the present invention can be used or administered as a mixture, for example in equal amounts, or individually, provided in sequence, or administered all at once. In providing a patient with CapD or PghP, the dosage of administered agent will vary depending upon such factors as the patient's age, weight, height, sex, general medical condition, previous medical history, etc.

Administration of the therapy could be performed orally or parenterally, or intravenously in amounts sufficient to enable the enzymes to degrade the organism's capsule. The administered protein can be in pure form, a fragment of the peptide, or a modified form of the peptide retaining enzymatic activity. One or more amino acids, not corresponding to the original protein sequence can be added to the amino or carboxyl terminus of the original peptide, or truncated form of peptide. Such extra amino acids are useful for coupling the peptide to another peptide, to a large carrier protein, or to a support. Amino acids that are useful for these purposes include: tyrosine, lysine, glutamic acid, aspartic acid, cysteine and derivatives thereof. Alternative protein modification techniques may be used e.g., NH₂-acetylation or COOH-terminal amidation, to provide additional means for coupling or fusing the peptide to another protein or peptide molecule or to a support.

The enzymes capable of degrading the capsule are intended to be provided to recipient subjects in an amount sufficient to effect a reduction in infection symptoms. An amount is said to be sufficient to “effect” the reduction of infection symptoms if the dosage, route of administration, etc. of the agent are sufficient to influence such a response. Responses to antibody administration can be measured by analysis of subject's vital signs.

Therapeutic compositions can be prepared according to methods known in the art. The present compositions comprise an amount of a recombinant CapD or PghP proteins or peptides as defined above, usually combined with a pharmaceutically acceptable carrier.

Pharmaceutically acceptable carriers include any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers; and inactive virus particles. Such carriers are well known to those of ordinary skill in the art. The carrier may be comprised of a saline solution, dextrose, albumin, a serum, or any combinations thereof.

The compositions typically will contain pharmaceutically acceptable vehicles, such as water, saline, glycerol, ethanol, etc. Additionally, auxiliary substances, such as wetting or emulsifying agents, pH buffering substances, preservatives, and the like, may be included in such vehicles.

Typically, the compositions are prepared as injectables, either as liquid solutions or suspensions; solid forms suitable for solution in, or suspension in, liquid vehicles prior to injection may also be prepared. The preparation also may be emulsified or encapsulated in liposomes. Solutions for infusion or injection may be prepared in a conventional manner, e.g. with the addition of preservatives such as p-hydroxybenzoates or stabilizers such as alkali metal salts of ethylenediamine tetraacetic acid, which may then be transferred into fusion vessels, injection vials or amplules. Alternatively, the compound for injection may be lyophilized either with or without the other ingredients and be solubilized in a buffered solution or distilled water, as appropriate, at the time of use. Non-aqueous vehicles such as fixed oils and ethyl oleate are also useful herein.

In cases where intramuscular injection is the mode of administration, an isotonic formulation can be used. Generally, additives for isotonicity can include sodium chloride, dextrose, mannitol, sorbitol and lactose. In some cases isotonic solutions such as phosphate buffered saline are preferred. Stabilizers include gelatin and albumin which may be included in the formulation. In some embodiments, a vasoconstriction agent is added to the formulation. The pharmaceutical preparations according to the present invention are provided sterile and pyrogen free.

The compounds of the present invention can be formulated according to known methods to prepare pharmaceutically useful compositions, whereby these materials, or their functional derivatives, are combined in admixture with a pharmaceutically acceptable carrier vehicle. Suitable vehicles and their formulation, inclusive of other human proteins, e.g., human serum albumin, are described, for example, in Remington's Pharmaceutical Sciences (16th ed., Osol, A. ed., Mack Easton Pa. (1980)). In order to form a pharmaceutically acceptable composition suitable for effective administration, such compositions will contain an effective amount of the above-described compounds together with a suitable amount of carrier vehicle.

Additional pharmaceutical methods may be employed to control the duration of action. Control release preparations may be achieved through the use of polymers to complex or absorb the compounds. The controlled delivery may be exercised by selecting appropriate macromolecules (for example polyesters, polyamino acids, polyvinyl, pyrrolidone, ethylenevinylacetate, methylcellulose, carboxymethylcellulose, or protamine sulfate) and the concentration of macromolecules as well as the method of incorporation in order to control release. Another possible method to control the duration of action by controlled release preparations is to incorporate the compounds of the present invention into particles of a polymeric material such as polyesters, polyamino acids, hydrogels, polylactic acid or ethylene vinylacetate copolymers. Alternatively, instead of incorporating these agents into polymeric particles, it is possible to entrap these materials in microcapsules prepared, for example, interfacial polymerization, for example, hydroxymethylcellulose or gelatin-microcapsules and poly(methylmethacylate)-microcapsules, respectively, or in colloidal drug delivery systems, for example, liposomes, albumin microspheres, microemulsions, nanoparticles, and nanocapsules or in macroemulsions. Such techniques are disclosed in Remington's Pharmaceutical Sciences (1980).

Administration of the compounds disclosed herein may be carried out by any suitable means, including parenteral injection (such as intravenous intraperitoneal, subcutaneous, or intramuscular injection), in ovo injection of birds, orally, or by topical application of the enzymes (typically carried in a pharmaceutical formulation) to an airway surface. Topical application to an airway surface can be carried out by intranasal administration (e.g., by use of dropper, swab, or inhaler which deposits a pharmaceutical formulation intranasally). Topical application to an airway surface can also be carried out by inhalation administration, such as by creating respirable particles of a pharmaceutical formulation (including both solid particles and liquid particles) containing the antibodies as an aerosol suspension, and then causing the subject to inhale the respirable particles. Methods and apparatus for administering respirable particles of pharmaceutical formulations are well known, and any conventional technique can be employed. Oral administration may be in the form of an ingestable liquid or solid formulation.

The treatment may be given to a subject in need of treatment and may include, but are not limited to, humans or ruminants, such as sheep and cows. The treatment is useful for a variety of illnesses caused by bacterial infections, to treat septicemia and deep tissue infection, illnesses caused by Staphylococcus epidermidis, and other illnesses involving bacterial organisms having a capsule which in part or in whole contains poly-γ-D-glutamic acid or poly-γ-DL-glutamic acid.

The treatment may be given in a single dose schedule, or preferably a multiple dose schedule in which a primary course of treatment may be with 1-10 separate doses, followed by other doses given at subsequent time intervals required to maintain and or reinforce the response, for example, at 1-4 days for a second dose, and if needed, a subsequent dose(s) after several days. Examples of suitable treatment schedules include: (i) 0, 1 day and 7 days, (ii) 0 and 7 days, and (iii) 0 and 14 days, or other schedules sufficient to elicit the desired responses expected to reduce disease symptoms, or reduce severity of disease.

Any dosage form employed should provide for a minimum number of units for a minimum amount of time. The concentration of the active unites of enzyme believed to provide for an effective amount of dosage of enzyme may be in the range of about 100 units/ml to about 500,000 units/ml of composition, preferably in the range of 1000 units/ml to about 100,000 units/ml, and most preferably from about 10,000 to 100,000 units/ml. The amount of active units/ml and the duration of time of exposure depend on the nature of infection, and the amount of contact the carrier allows the enzyme to have. It is to be remembered that the enzyme works best when in a fluid environment. Hence, effectiveness of the enzyme is in part related to the amount of moisture trapped by the carrier. For the treatment of septicemia, there should be a continuous intravenous flow of therapeutic agent into the blood stream. The concentration of enzyme for the treatment of septicemia is dependent on the seriousness of the infection.

In order to accelerate treatment of the infection, the therapeutic agent may further include at least one complementary agent which can also potentiate the bactericidal activity of the enzyme. The complementary agent can be penicillin, ciprofloxacin (used to treat anthrax infection), or vancomycin (used to treat S. epidermidis infection), synthetic penicillins bacitracin, methicillin, cephalosporin, polymyxin, cefaclor. Cefadroxil, cefamandole nafate, cefazolin, cefixime, cefmetazole, cefonioid, cefoperzone, ceforanide, cefotanme, cefotaxime, cefotetan, cefoxitin, cefpodoxime proxetil, ceftazidine, ceftizoxime, ceftriaxone, ceftriaxone moxalactam, cefuroxime, dihydratecephalothin, moxalactam, loracarbef, mafate, chelating agents and any combinations thereof in amounts which are effective to synergistically enhance the therapeutic effect of the enzyme.

The present invention also provides kits which are useful for carrying out the present invention. The present kits comprise a first container means containing the enzymes of the invention. The kit also comprises other container means containing solutions necessary or convenient for carrying out the invention. The container means can be made of glass, plastic or foil and can be a vial, bottle, pouch, tube, bag, etc. The kit may also contain written information, such as procedures for carrying out the present invention or analytical information, such as the amount of reagent contained in the first container means. The container means may be in another container means, e.g. a box or a bag, along with the written information.

The contents of all cited references (including literature references, issued patents, published patent applications, and co-pending patent applications) cited throughout this application are hereby expressly incorporated by reference.

All publications, including, but not limited to patents and patent applications, cited in this specification are herein incorporated by reference as if each individual publication were specifically and individually indicated to be incorporated by reference herein as though fully set forth.

The invention is further described in detail to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided therein.

The following Materials and Methods were used in the Examples below.

Bacterial strains and spore preparation. B. anthracis Ames (pX01⁺, pX02⁺) and ΔAmes (pX01⁻, pX02⁺) (United States Army Medical Research Institute of Infectious Diseases collection) were cultured in brain heart infusion (BHI) broth (Becton Dickinson and Co., Sparks, Md.) at 37° C. with 0.8% bicarbonate and 5% carbon dioxide. B. anthracis spores were generated as previously described (Chabot et al., 2004, Vaccine 23, 43-47).

Cloning and expression of PhgP and CapD

The open reading frame of capD excluding the signal sequence was amplified by PCR and cloned into pET15b as an XhoI HindIII fragment: forward primer: 5′-GTC GCT CGA GTC TTT CAA TAA AAT AAA AGA CAG TGT TA-3′ (SEQ ID NO:5) and reverse primer: 5′-GCG GCG AAG CTT CTA TTT ATT TGA TTT CCA AGT TCC ATT CTC TCT GCC-3′(SEQ ID NO:6). The open reading frame of the pghP gene was amplified from φNIT1DNA with the following primers: forward primer: 5′-GCG GCG CAT ATG GCA CAA ACA GAC ACA TAT CCA AAT ATT GAA GCA-3′(SEQ ID NO:7) and reverse primer: 5′-GCG GCG GGA TCC TTA GCC ATA ATA CTC TGC CTC TGC TTC TTT AAT-3′(SEQ ID NO:8). The PCR product generated was cloned into the pET15B expression vector (Novagen) as a BamHI NdeI fragment.

Protein purification. The capD gene encoding amino acids 28-528 was expressed in pET 15b as an XhoI HindIII fragment (EMD Biosciences, San Diego, Calif.). An additional construct of the capD gene encoding amino acids 43-528 was cloned into pTYB12 as an EcoRI NdeI fragment and expressed using the IMPACT™ Protein Purification System which allows over-expression of a target protein as a fusion to a self-cleavable affinity tag (New England BioLabs, Beverly, Mass.). This construct retained enzyme activity and was found to give a higher yield of enzyme and was used for all animal experiments. Recombinant protein was expressed and purified according to the manufacturer's instructions and stored in phosphate buffered saline, pH 7.4 (PBS). For B. anthracis Ames challenges, endotoxin was removed from CapD preparations with an EndoTrap^{R} Red column (Cambrex Corp., Walkersville, Md.) according the manufacturer's instructions.

Human neutrophils. Human neutrophils were purified from normal unvaccinated volunteers by

Ficoll-Hypaque density gradient centrifugation followed by dextran sedimentation (Kuhns et al., 2001, J. Immunol. 167, 2869-2878). Purified neutrophils were used to examine CapD mediated neutrophil killing. Briefly, purified human neutrophils were resuspended in Dulbecco's Modified Eagle Medium (DMEM) containing 10% heat inactivated fetal bovine serum. Neutrophils were mixed with encapsulated B. anthracis bacilli at an effector to target ratio of 50:1 (6×10⁶ neutrophils/ml, 1×10⁵ bacilli/ml) and incubated for 2 h at 37° C. on an Eppendorf tube rotator. Following incubation, bacilli viability was measured by serial dilution in water and plating on Luria Bertani agar. To examine the effect of toxin on neutrophil bactericidal activity, neutrophils were incubated with combinations of PA (1 μg/ml), LF (0.3 μg/ml), and EF (0.3 μg/ml) for 2 h at 37° C. on an Eppendorf tube rotator. Encapsulated bacilli, 10% normal human serum (Type AB⁺), and CapD (20 μg/ml) or PBS were then added and the mixtures rotated for an additional 2 or 5 h. Bacterial viability was then measured by serial dilution and plating.

Glutamylase assay. Native capsule from B. subtilis and B. anthracis was purified as described (Chabot, Scorpio et al. 2004, Vaccine 23, 43-7) and digested with 10 fold dilutions of purified recombinant CapD or PghP for one hour at 37° C. Each reaction contained 1.5 ul capsule, 15.5 ul PBS, 1 ul 2 mM ZnSO4, and 2 ul (1 ug) enzyme. No ZnSO4 was added to the reaction containing CapD. Following the reaction, an equal volume (20 ul) of 2×SDA tricine sample buffer was added to each sample and the degradation products analyzed on a 10% SDS tricine PAG. Synthetic gamma poly-glutamate peptides (AnaSpec Inc. San Jose, Calif.) were also tested as substrates for CapD and PghP. Peptides consisting of 30 D- or L-glutamate residues or 15 D- or 15 L-residues were digested with enzyme for 1 hour at 37° C. and analyzed as described for native capsule.

Macrophage phagocytosis. Murine RAW 264.7 macrophages were cultured and phagocytosis assays were performed as previously described (Chabot et al., 2004, supra). Purified PghP or CapD was added to a final concentration of 20 ug/ml and erythromycin was added to a final concentration of 0.5 ug/ml. The phagocytic index was calculated by determinig the average number of bacilli adhered to each macrophage.

Animals. Female Balb/c mice (6-8 weeks) were used for ΔAmes bacillus challenge experiments. All other experiments were performed with Female Swiss Webster mice (6-8 weeks). Mice were obtained from the National Cancer Institute, Fort Detrick (Frederick, Md.).

Human neutrophil and serum bactericidal assays. Human neutrophils were resuspended in DMEM at a concentration of 2×10⁷/ml. Newly germinated, encapsulated B. anthracis were resuspended in DMEM at 1×10⁷/ml and treated with either PBS or 20 ug/ml CapD or PghP and incubated for 10 minutes at 37° C. Neutrophils were adjusted to 5×10⁶/ml in a 1.5 ml Eppendorf tube and enzyme treated bacilli were added to 1×10⁵/ml for an effector to target ratio of 50:1. Normal human serum was added to 10% as a source of complement, giving a final volume of 550 ul. An aliquot was immediately removed and bacterial viability measured by serial dilution in water and plating on LB agar. The remaining sample was mixed on an Ependorf tube rotator at 37° C. Bacterial viability was measured at 2 and 3 hours. Experiments were performed with and without erythromycin. Serum bactericidal activity was measured by incubating newly germinated, encapsulated bacilli at 1×10⁶/ml in normal human serum, untreated or heat inactivated.

Bacillus challenge infection model. B. anthracis Ames and ΔAmes spores were used to inoculate BHI broth containing 0.8% bicarbonate. The cultures were grown at 37° C./5% CO₂ overnight with shaking. The cultures were then diluted 1:1000 in fresh BHI and grown for an additional 6 hours until they were approximately 10⁷ CFU/ml. Encapsulated bacilli were harvested by centrifugation and washed twice in phosphate buffered saline, pH 7.4. The chain length of Ames at this time was 5-10 bacilli per chain and 10-20 bacilli per chain for ΔAmes. Bacilli were treated with CapD (20 μg/ml) for 10 minutes, 37° C. As a control, CapD was heat inactivated by incubating the enzyme at 75° C. for 30 min. PBS was used as the control in the ΔAmes challenge experiment. To avoid precipitation during heating, CapD in PBS was diluted 1:10 in water, heat inactivated and then adjusted back to 1×PBS by adding 10×PBS. Following incubation of bacilli with CapD, normal Swiss Webster mouse serum was added to the suspensions to a final concentration of 10%. Mice that had been administered by intraperitoneal (i.p.) injection 1 ml of a 2% starch solution 6 hr earlier were challenged (200 μl i.p.) with the treated bacilli suspensions.

Spore challenge infection model. B. anthracis Ames or ΔAmes spores were washed twice with water for injection and heat shocked at 65° C. for 40 min. Swiss Webster mice were infected i.p. with 200 μl of B. anthracis spore suspension. Purified CapD was administered i.p. (200 μl in PBS) concurrently with spore challenge, or i.p. and intravenously (i.v.) 30 hr after challenge. For all experiments, intravenous administration was via the tail vein in a volume of 200 μl.

Statistics. Differences in mouse survival rates were analyzed with Fisher's Exact test.

Survival curves were performed with Kaplan Meier survival analysis with log-rank test.
Delays in mean time to death were determined with Wilcoxon rank-sum test.

### EXAMPLE 1

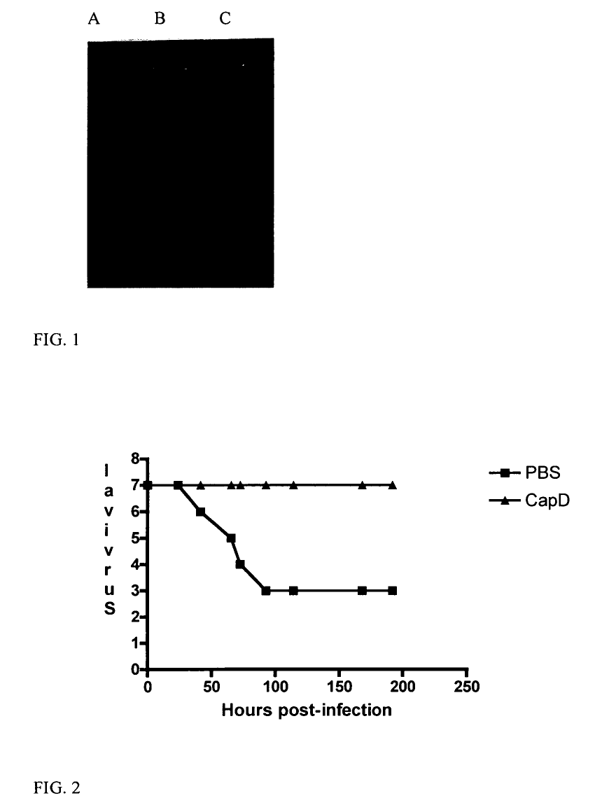

Recombinant CapD. The open reading frame of capD excluding the signal sequence is from amino acids 28-528. We originally cloned this segment of capD for use in neutrophil killing assays but consistently observed low yields following expression and purification. An additional construct of amino acids 43-528 was cloned into pTYB12 which yielded approximately 10 fold higher expression levels and higher purity CapD. To evaluate the polyglutamate capsule degrading activity of CapD 43-528, purified B. anthracis capsule was incubated for 1 h at 37° C. with PBS, CapD 28-528 or CapD 43-528 (50 μg/ml) and the degraded capsule products analyzed on a 1% agarose gel. FIG. 1 shows that the two forms of CapD have similar enzymatic activities. Additionally, the two enzyme forms mediated similar levels of neutrophil killing in a human neutrophil killing assay (see below). The higher yield and similar enzymatic activity of CapD 43-528 prompted us to use this construct for all animal experiments.

### EXAMLE 2

Hydrolysis of capsule with purified CapD and PghP. Encapsulated B. anthracis ΔAmes bacilli were treated with 0.5 ug of recombinant CapD or PghP and visualized by phase contrast microscopy. Capsule was cleaved from the bacilli within 5 minutes (data not shown). Similar results were seen with wild type B. anthracis Ames. PghP appeared to degrade B. anthracis capsule faster but CapD was more efficient in inhibiting re-growth of capsule during prolonged incubation. To determine the extent of degradation, purified capsule from B. anthracis Ames and B. subtilis was digested with purified PghP or CapD for 2 house at 37° C. and examined by SDS-PAGE on a 10% SDS Tricene gel. Complete hydrolysis of B. anthracis capsule was not observed with any amount of PghP tested. By contrast, B. anthracis capsule was digested to completion with 35 ug/ml CapD, although this activity appears to be significantly less than the activity of PghP on B. subtilis capsule. The capsule from B. subtilis was digested to completion with 3.5 ug/ml PghP and was significantly reduced in size when treated with as little as 0.035 ug/ml enzyme. By comparison, hydrolysis of B. anthracis capsule resulting in a similar pattern and size seen with 0.035 ug/ml enzyme reacted with B. subtilis capsule was only seen with 35 ug/ml enzyme suggesting the efficiency of digestion of B. anthracis capsule by PghP is only 0.1% of its activity on B. subtilis capsule.

To examine the relative inefficiency of PghP in degrading B. anthracis capsule, synthetic 30mers of D- or L-polyglutamate were digested with purified enzyme. No hydrolysis of the D-polyglutamate was seen but complete hydrolysis of L- and 30mers containing both D- and L-was observed (data not shown). These results suggest that the efficiency of D-hydrolysis may be due to poor binding of enzyme to D-polyglutamate and not lower enzymatic activity since PghP is equally effective against mixtures of D- and L-polyglutamate from capsule grown under various Mn++ concentrations (Kimura and Itoh, 2003, Appl. Environ. Microbiol. 69, 2491-2497). Thus, the lower activity of recombinant PghP against B. anthracis capsule may be related to a lower binding affinity to D-polyglutamate.

### EXAMPLE 3

Opsonization of encapsulated bacilli. We next examined the effect of enzymatic treatment of encapsulated B. anthracis on phagocytosis by macrophages. Encapsulated B. anthracis bacilli were treated with purified CapD or PghP as described in the Materials and Methods. To examine the effect of capsule removal on phagocytosis, encapsulated bacilli were treated with recombinant enzyme, pipetted onto either peritoneal or RAW 264.7 murine macrophages adhered to glass cover slips and incubated at 37° C. After a washing step, the macrophages were stained with Wright Giemsa stain and visualized by microscopy. Bacilli adherent to macrophages were counted over several fields of view constituting several hundred macrophages to determining a phagocytic or opsonic index, defined as the number of adhered bacilli per macrophage. Both enzymes had a significant effect on the phagocytic index of encapsulated bacilli. The phagocytic indices after treatment of B. anthracis with CapD and PghP were significantly higher than the PBS control in the presence of 0.5 ug/ml erythromycin while only CapD showed an effect in the absence of erythromycin (data not shown). Bacilli treated with purified enzyme were significantly more adherent to macrophages that untreated bacilli. The extent of phagocytosis observed in the presence of recombinant CapD, however, was higher than with PghP and similar to that seen with non-encapsulated bacilli germinated from spores in the absence of bicarbonate and CO₂ (data not shown). This is consistent with the degree of degradation of purified capsule observed with CapD compared to PghP treatment. While significant levels of bacterial attachment to the macrophages were observed, relatively few bacilli were seen engulfed by the RAW 264.7 macrophages. This may be due to the short duration of the incubation. Experiments with human neutrophils and peritoneal macrophages, however, revealed significant uptake of bacilli following treatment with enzyme. Taken together, the data indicate that capsule removal opsonizes encapsulated bacilli for uptake by phagocytes.

### EXAMPLE 4

Enzyme mediated killing of encapsulated bacilli. We next wanted to examine the effects of recombinant CapD and PghP on phagocitic killing by freshly isolated human neutrophils or mouse peritoneal macrophages. Phagocytic cells were mixed with newly germinated, encapsulated B. anthracis bacilli in the presence of enzyme and bacterial viability measured by serial dilution and plating. Purified PghP and CapD facilitated neutrophil and macrophage mediated killing of encapsulated B. anthracis (data not shown). Neutrophil killing activity was measured in the presence and absence of a sub-lethal concentration of erythromycin (0.5 ug/ml) which was added to inhibit capsule regeneration during the incubation. The level of human neutrophil bactericidal activity in the presence f purified CapD was significantly higher than that observed for PghP and was independent of the presence of erythromycin. The CapD enzyme mediated killing of greater than 99% of encapsulated bacilli with or without erythromycin while PghP mediated killing of greater than 95% of bacilli in the presence of erythromycin. Neutrophil bactericidal activity was complement dependent with little bactericidal activity observed in the presence of heat inactivated serum. A 2 log drop in bacterial viability was observed by 2 hours post-infection suggesting vegetative bacilli are rapidly killed when phagocytosed by neutrophils. Additionally, incubation of encapsulated bacilli in serum alone did not result in significant loss of viability. This was true whether or not the serum was heat inactivated suggesting that the effect of complement on neutrophil killing was due to complement mediated opsonization of bacilli or complement mediated activation of the neutrophil oxidative burst. The level of enzyme mediated adherence to macrophages, however, was not dependent on the presence of complement suggesting complement may be more important to activate neutrophils than to promote phagocytosis of the organisms.

To examine the concentration of CapD required for neutrophil killing, the enzyme was serially diluted in PBS and used to treat newly germinated, encapsulated bacilli for neutrophil bactericidal assays. The minimum concentration of CapD necessary to facilitate efficient neutrophil killing (approximately 2 logs) of encapsulated bacilli was determined to be approximately 1 ug/ml (data not shown). As little as 0.25 ug/ml was sufficient to facilitate killing of greater than 90% of bacilli. The neutrophil bactericidal assays were performed at an E:T of 50:1. However, even at a 1:1 ratio, neutrophils actively engulf and kill encasulted bacilli in the presence of CapD resulting in a 2 log drop in bacterial viability. Examining the effect of CapD concentration on the presence of capsule by India ink staining revealed that as little as 0.035 ug/ml was sufficient to visibly decrease the amount of capsule on the surface of bacilli (data not shown).

Macrophage mediated killing was also performed with CapD and PghP. While the level of PghP mediated macrophage bactericidal activity was significantly lower than that observed with CapD, both enzymes were shown to mediate macrophage killing of encapsulated bacilli in the presence of erythromycin. Collectively, our data suggest enzymatic capsule removal facilitates complement dependent phagocytic killing of encapsulated B. anthracis bacilli.

### EXAMPLE 5

CapD treatment protects against B. anthracis bacillus challenge. To demonstrate the effect of CapD treatment on the virulence of B. anthracis bacilli, we designed an experiment to expose CapD treated bacilli to host neutrophils at the time of challenge with the hypothesis that this would mimic the environment of anthrax bacilli after dissemination into the blood. Sterile irritants such as starch can be used as a chemoattractant to elicit influx of polymorphonuclear leukocytes to the i.p. space by 4 hr post injection (Welkos et al., 1989, Microb. Pathog. 7, 15-35). Swiss Webster mice were administered i.p. 1 ml of a 2% starch solution. Six hr later the mice were challenged with B. anthracis bacilli incubated for 10 min with normal CapD, heat inactivated CapD or PBS. Mice challenged with 1000 CFU ΔAmes bacilli (approximately 10,000-20,000 bacilli) treated with CapD survived (7/7) while 3/7 mice given PBS survived (FIG. 2, P=0.035). An experiment was performed to confirm no loss of bacterial viability during incubation of bacilli in PBS containing 10% serum and CapD but in the absence of neutrophils. This suggests that bacilli treated with CapD were killed in vivo after infection.

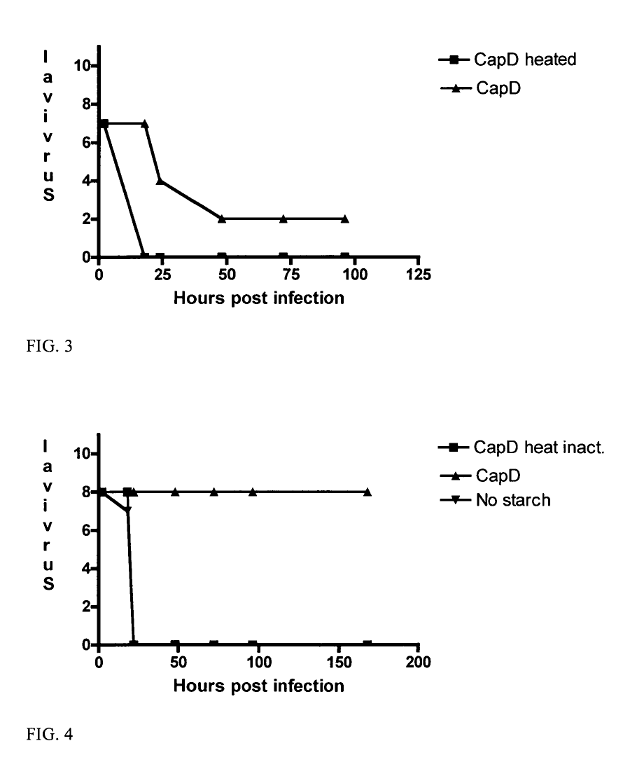

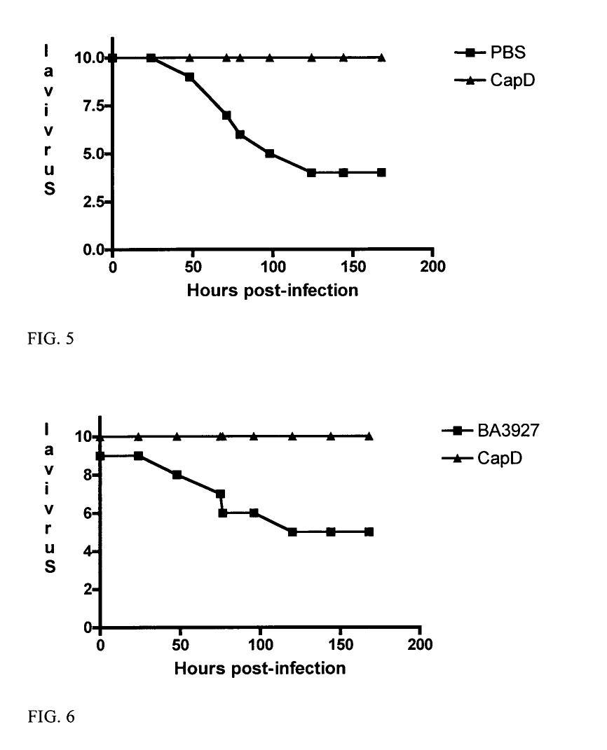

To examine the effect of CapD treatment on fully virulent B. anthracis, two experiments were performed with B. anthracis Ames. In the first experiment, 7 mice were challenged with 4,000 CFU (approximately 20,000 bacilli) treated with either CapD or heat inactivated CapD. All 7 mice challenged with bacilli treated with heat inactivated CapD died within 18 hr. Of the mice given CapD treated bacilli, all seven survived for at least 28 hr, 3 died by 42 hr and 2 died by 66 hr. Two mice survived for the duration of the experiment. This represents a significant delay in mean time to death (FIG. 3, P=0.0099) as well as significant difference in survival curve (P=0.0003). In the second experiment, 8 mice per group were challenged with 500 CFU (approximately 2500 bacilli) treated with normal or heat inactivated CapD. An additional control group of mice that were not given 2% starch prior to challenge was included. This group was challenged with bacilli treated with PBS. All 8 mice challenged with bacilli treated with heat inactivated CapD died between 18-22 hr post-infection. Similarly, all 8 mice that were not given starch prior to infection and challenged with bacilli treated with PBS died between 18-22 hr. The nearly identical kinetics of infection and time to death observed for these two groups indicates that fully encapsulated bacilli are resistant to neutrophil killing in the host. By contrast, all 8 mice administered bacilli treated with CapD survived for the duration of the experiment (FIG. 4) suggesting the infecting dose was killed by neutrophils present at the site of infection. These results demonstrate that enzymatic capsule removal facilitates killing of B. anthracis bacilli in the host and suggests that fully virulent B. anthracis bacilli are highly susceptible to killing by host innate immunity cells following removal of the polyglutamate capsule.

### EXAMPLE 6

Post-exposure CapD treatment. The potential of CapD as a post-exposure therapy was evaluated in Swiss Webster mice following challenge with B. anthracis ΔAmes heat shocked spores. In the initial experiment, mice were challenged with an i.p. injection of 1500 ΔAmes spores containing 1.33 mg recombinant CapD or PBS, 10 mice per group. At 24 hr post-infection, mice were given an additional i.p. injection of 533 μg CapD and monitored for mortality. Six of 10 mice in the CapD group survived compared with 3 of 10 in the PBS group but this did not meet statistical significance (P=0.18). We therefore repeated the experiment with some alterations; mice were challenged i.p. with 2000 spores concurrently with 400 μg CapD or PBS and subsequently given i.p. 400 μg CapD and i.v. 400 μg CapD or PBS at 30 hr post-infection. Mice given CapD all survived (10/10) compared with 4 of 10 surviving in the PBS group (FIG. 5, P=0.005) suggesting intravenous delivery of CapD was more effective than i.p. delivery in affording protection following spore challenge.

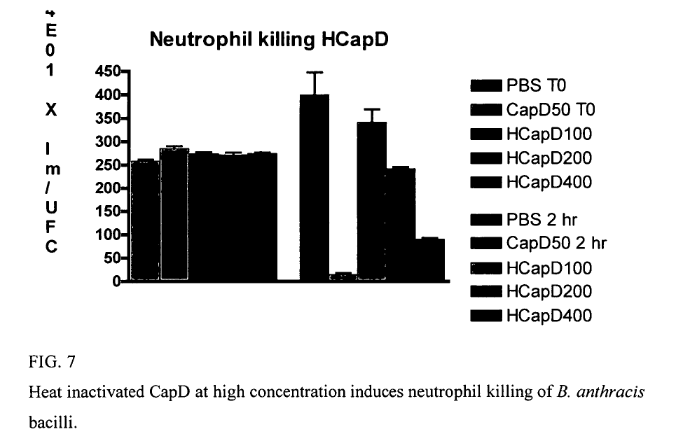

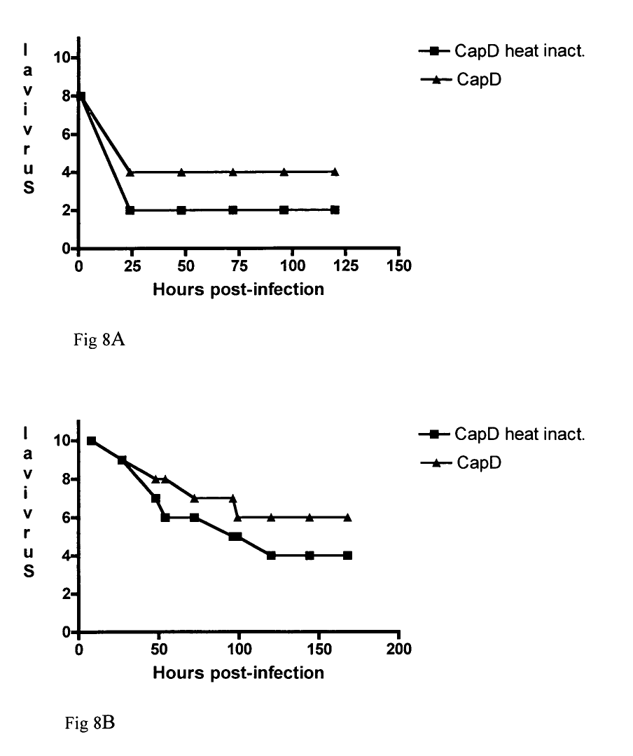

The effect of CapD on survival when given post-exposure was then examined. For these experiments, mice were not administered CapD at the time of infection but rather given i.p. 400 μg and i.v. 400 μg 30 hr after spore challenge. In the first experiment, CapD was compared with an irrelevant protein, a basic membrane lipoprotein, BA3927. The open reading frame of BA3927 was cloned into pTYB12 and purified by the same method as CapD. Mice were challenged i.p. with 5000 ΔAmes spores and administered i.p. and i.v. doses (400 μg) of either CapD or BA3927 at 30 hr. Ten of 10 mice in the CapD group survived, compared with 5 of 9 surviving in the group administered BA3927 (FIG. 6, P=0.035) suggesting protection was CapD specific. To determine the effect of enzyme inactivation on protection from challenge, mice were administered CapD or heat-inactivated CapD 30 hr after challenged with 2000 ΔAmes spores. Surprisingly, all mice in each group (10/10) survived. To examine this, we performed macrophage phagocytosis and neutrophil killing assays with normal and heat inactivated CapD. Interestingly, heat inactivated CapD had a dose dependent effect on both the macrophage phagocytic index and killing of B. anthracis bacilli by human neutrophils (FIG. 7). Treatment of encapsulated bacilli with heat inactivated CapD at 400 μg/ml resulted in a 7-fold drop in viability compared with the PBS control (FIG. 8). Similarly, CapD treatment with 250 μg/ml resulted in a macrophage phagocytic index of 3.5+/−0.3 compared with 0.28+/−0.046 in the PBS control. No degra-dation of B. anthracis capsule was observed when incubated with heat inactivated CapD, even at 400 μg/ml suggesting the heated CapD may lose its enzymatic activity but retain its capsule binding function. Binding of the heat inactivated CapD to surface associated capsule may increase recognition by phagocytic cells, essentially opsonizing the encapsulated bacilli.

Two experiments were performed with the fully virulent Ames strain in the spore challenge model. In both experiments, normal or heat-inactivated CapD was administered i.p. and i.v. 30 hr post-infection. We did not observe statistically significant protection in either experiment, although in both cases, there was a higher rate of survival in the CapD treated group. The combined results are summarized in FIG. 8 and suggest that adminis-tration of enzymatically active CapD resulted in better protection than heat-inactivated CapD although not statistically significant (P=0.15). Taken together, the data suggest that post-exposure administration of CapD resulted in increased survival in mice.

### EXAMPLE 7

Toxin does not inhibit neutrophil bactericidal activity. To examine the effect of toxin on neutrophil bactericidal activity, purified human neutrophils were incubated in DMEM for 2 h or 5 h, 37° C. in an Eppendorf tube rotator in the presence of PA, LF, EF or combinations of these. Encapsulated bacilli, serum, and CapD or PBS were then added. The final concentration of neutrophils was 5×10⁶/ml and bacilli were 1×10⁵/ml. The mixtures were rotated for an additional 2 h and bacterial viability was measured by serial dilution and plating. As illustrated in Table 1, pre-incubation with toxin had no effect on neutrophil bactericidal activity for either pre-incubation time. All combinations of toxin components tested resulted in greater than 99% reduction in bacterial viability. These results demonstrate that while the toxins reportedly have significant effects on neutrophil functions, including chemotactic migration, actin polymerization and NADPH oxidase activity, they do not affect bactericidal activity.

TABLE 1
Neutrophil bactericidal activity is not affected
by pre-incubation with toxins or capsule.

| | % survival | % survival |
|---|---|---|
| Treatment | 2 hr | 5 hr |
| None | 111 | |
| PA | 0.31 | 0.11 |
| LF | 0.23 | 0.18 |
| EF | 1 | 0.32 |
| LT | 0.46 | 0.29 |
| ET | 0.62 | 0.18 |
| Capsule | 2.9 | 0.25 |
| CapD | 0.54 | 0.1 |
| LT ET | 0.15 | 0.14 |
| PA63 LT | ND | 0.15 |

. Human neutrophils were pre-incubated with toxins or capsule as described in the Materials and Methods. Following incubation, serum, encapsulated bacilli, and CapD were added and the mixtures incubated an additional 2 h. Bacterial viability (% survival) was measured by serial dilution and plating on LB agar.

### EXAMPLE 8

Stability of CapD. The activity of CapD following incubation in serum was examined. Enzyme was incubated in 100% Swiss Webster mouse serum or type AB⁺ human serum and assayed for activity by mixing the serum with encapsulated B. anthracis bacilli followed by viewing the capsule by India ink stain and wet mount. The activity of CapD at an initial concentration of 40 μg/ml was greatly diminished after 4 hr incubation in mouse serum. This decrease in activity could be rescued by addition of protease inhibitors to the serum suggesting serum proteases degrade the enzyme during incubation. By contrast, the activity of the enzyme after 4 hr incubation in human serum was similar to its activity after 4 hr in PBS suggesting lower proteolytic activity of human serum compared with mouse serum. Interestingly, CapD 43-528 appeared to be more sensitive to serum inactivation than CapD 28-528. We used CapD 43-528 for all mouse studies due to its higher yield following expression in E. coli; however it may be inferior to CapD 28-528 in affording protection if it is significantly more sensitive to serum proteolysis.

### Discussion

Fully virulent Bacillus anthracis bacilli expend a significant amount of metabolic energy producing a polyglutamate capsule during infection. The value of the capsule to its survival and replication in a host is well demonstrated and is thought to be related to a potent antiphagocytic property. Recent work has shown that immune responses to the capsule can afford protection from infection by targeting anthrax bacilli to phagocytes and probably in particular to cells of the innate immune system (Chabot, 2004, supra). In addition to opsonizing anthrax bacilli with capsule specific antibodies, we have shown it is possible to target the bacilli to phagocytes by enzymatically removing the capsule which results in a highly efficient level of phagocytosis. While B. anthracis spores are relatively resistant to phagocytic killing (unpublished observations), the vegetative form of the organism is highly susceptible to killing by neutrophils (Mayer-Scholl et al., 2005, supra; Scorpio et al., 2005, supra). And although neutrophils are reportedly not important during the early, spore stage of infection (Cote et al., 2006, Infect. Immun. 74, 469-480), they may be very important late in infection during which time the bacillus form predominates and is exposed to circulating neutrophils. As mentioned above, enzyme treatment to remove bacterial capsule has been successfully used to cure existing infections with pneumococci and E. coli (Avery and Dubos, 1931, J. Exp. Med. 54, 73-89; Mushtaq, 2005, supra) in mouse models of infection. In both studies, the authors attributed the observed protection to increased levels of phagocytosis resulting from capsule removal in vivo. Two experimental methods were employed in our studies: concurrent i.p. administration of CapD with a bacillus challenge and post-exposure intravenous and i.p. administration of CapD after a spore challenge. Significant CapD mediated protection was observed in the bacillus challenge model with either ΔAmes or the fully virulent Ames strain as well as the spore challenge model with ΔAmes. Significant protection was not achieved when CapD was administered after spore challenge with B. anthracis Ames, although the results were suggestive of partial protection. A significant obstacle in our approach is the problem of maintaining enzyme concentration in the blood. We observed a significant decrease in enzyme activity after only 4 h incubation in mouse serum and could not detect activity by India ink staining of encapsulated bacilli incubated in serum from a mouse bled 3 h after i.v. administration of CapD. Additionally, we only administered CapD once following infection. This combination of factors may explain the lack of significant protection observed in the Ames spore infection model.

We propose that enzymatic stripping of capsule from anthrax bacilli in vivo sensitizes the bacilli to phagocytic killing and eventual clearance of the organisms from the blood and infected organs. The higher level of survival in mice treated with CapD both during and after infection may thus be a result of enzyme mediated phagocytosis of capsule producing bacilli that are rapidly killed. In both the bacillus challenge and spore infection models, we attempted to design the experiment to maximize exposure of encapsulated bacilli to CapD while in proximity to neutrophils, either in the blood or the i.p. cavity. While the blood normally has a circulating neutrophil concentration of approximately 5×10⁶/ml, we injected starch into the i.p. cavity to stimulate migration of neutrophils to the site of infection to simulate late stage infection. It is possible that other factors such as antimicrobial peptides in serum that have higher bactericidal activity on decapsulated bacilli may contribute to protection. However, even non-capsule producing strains of B. anthracis are highly resistant to serum killing. Because neutrophils are highly efficient in killing bacteria and have higher bactericidal activity against B. anthracis than other leukocytes such as macrophages (unpublished observations), we hypothesize that the protection observed is due to neutrophil killing of B. anthracis bacilli.

The threat of antibiotic or vaccine resistant B. anthracis emerging as a potential biological weapon increases the likelihood that alternative treatments may be necessary. A treatment such as CapD that targets B. anthracis bacilli to neutrophils would presumably be effective against virtually any antibiotic or vaccine resistant strain due to its unique mechanism of action. Additionally, because neutrophils appear to retain bactericidal activity even after exposure to toxin, CapD may facilitate neutrophil killing of circulating bacilli when administered post-infection and thus represent an effective therapeutic treatment. Furthermore, it has been proposed that antibiotic treatment of systemic anthrax is often unsuccessful due to release of toxin from circulating bacteria that lyse following exposure to antibiotics. By targeting the bacteria for ingestion and killing by host cells, CapD would facilitate removal of toxin (contained in circulating bacilli) from the blood stream that may circumvent this problem.

Recombinant enzymes generally have a poor pharmacokinetic profile and only a few such as streptokinase and plasminogen activator for thrombolytic blood clot therapy have been approved for human use. As such, development of small molecule inhibitors that target capsule synthesis or regulation may prove to be a better strategy for developing an anthrax specific antibiotic. A treatment such as CapD, if effective, however, could be used as a last line of defense treatment for anthrax resulting from infection with genetically engineered strains that are resistant to traditional or other small molecule antibiotics.

What is claimed is:

1. An isolated recombinant capsule depolymerase, CapD.

2. The CapD of claim 1 having the amino acid sequence specified in SEQ ID NO:2.

3. The CapD of claim 2 encoded by the polynucleotide sequence specified in SEQ ID NO:1.

4. A vector comprising the polynucleotide fragment of claim 3.

5. The vector of claim 4 wherein said vector is pTYB12capD.

6. The vector of claim 4 wherein said vector is pMALcapD.

7. A host cell transformed with the vector of claim 5.

8. The host cell of claim 7 wherein said cell is prokaryotic.

9. A host cell transformed with the vector of claim 6.

10. The host cell of claim 9 wherein said host cell is prokaryotic.

11. A method for producing recombinant CapD protein comprising:
growing a host cell according to claim transformed with a vector expressing CapD in a suitable culture medium,
causing expression of said vector sequence as defined above under suitable conditions for production of soluble protein and,
lysing said transformed host cells and recovering said CapD.

12. The method of claim 10 wherein said host is prokaryotic.

13. A composition for degrading a poly-γ-D-glutamic polymer, said composition comprising recombinant CapD.

14. A method for degrading a poly-γ-D-glutamic polymer comprising bringing said polymer into contact with the composition of claim 13 in an amount sufficient to produce said degradation.

15. A method for reducing symptoms of an infection in a subject, said infection produced by an organism having a capsule comprising a poly-γ-D-glutamic polymer, said method comprising introducing into said subject a composition according to claim 13, in an amount effective for degrading said capsule, such that said symptoms are reduced.

16. The method of claim 14 wherein said organism is B. anthracis.

17. The method of claim 14 wherein said composition further comprises an agent which can potentiate bactericidal activity of CapD.

18. The method of claim 17 wherein said agent is an antibiotic.

19. The method of claim 18 wherein said antibiotic is ciprofloxacin.

20. An isolated recombinant poly-γ-glutamate hydrolase, PghP.

21. The PghP of claim 20 having the amino acid sequence specified in SEQ ID NO:3.

22. The PghP of claim 21 encoded by the polynucleotide sequence specified in SEQ ID NO:4.

23. A vector comprising the polynucleotide fragment of claim 22.

24. The vector of claim 23 wherein said vector is pET15 bpghp.

25. A host cell transformed with the vector of claim 24.

26. The host cell of claim 25 wherein said cell is prokaryotic.

27. The host cell of claim 26 wherein said host cell is prokaryotic.

28. A method for producing recombinant PghP protein comprising:
growing a host cell according to claim transformed with a vector expressing PghP in a suitable culture medium,
causing expression of said vector sequence as defined above under suitable conditions for production of soluble protein and,
lysing said transformed host cells and recovering said PghP.

29. The method of claim 28 wherein said host is prokaryotic.

30. A composition for degrading a poly-γ-DL-glutamic polymer, said composition comprising recombinant PghP.

31. A method for degrading a poly-γ-DL-glutamic polymer comprising bringing said polymer into contact with the composition of claim 30 in an amount sufficient to produce said degradation.

32. A method for reducing symptoms of an infection in a subject, said infection produced by an organism having a capsule comprising a poly-γ-DL-glutamic polymer, said method comprising introducing into said subject a composition according to claim 30, in an amount effective for degrading said capsule, such that said symptoms are reduced.

33. The method of claim 32 wherein said organism is Staphylococcus epidermidis.

34. The method of claim 33 wherein said composition further comprises an agent which can potentiate bactericidal activity of PghP.

35. The method of claim 34 wherein said agent is an antibiotic.

36. The method of claim 35 wherein said antibiotic is vancomycin.


SEQUENCE LISTING (8 sequences)

SEQ ID NO: 1 - DNA, 1505 bp, B. Anthracis
tctttcaataaaataaaagacagtgttaagcaaaaaattgatagtatgggtgataaaggaacttatggagtgagtgcctctcacccccttgcggttgaggaaggtatgaaagtattaaagaacggtggaagtgcagtagatgcagcgattgtggtctcatatgttttaggcgttgtagaactgcatgcctcaggaataggtgggggcggtggaatgctcattatatctaaagataaagaaacctttattgattatcgtgaaacaactccgtactttacaggaaaccaaaagccacatattggagtacccggatttgtggctggaatggagtatattcatgataattatggttcattaccgatgggtgagttattacaaccagccattaattatgcggaaaaagggttcaaggtagatgattccttaacaatgcgattagaccttgcgaagccacgtatttattctgataagctaagtatcttctatccgaatggtgaacctattgaaactggagaaacacttatccagacagatttagcgagaaccttaaagaagattcaaaaagaaggggctaaaggcttttatgaaggaggagtcgctagggcaatcagtaaaactgcaaaaatatcgttagaagatataaaaggatataaagtagaggtacgtaaaccagtaaaaggtaactacatgggatatgatgtttataccgctccaccacctttttcaggagttactttattacaaatgttgaaattagctgaaaagaaagaagtatataaagatgtagatcatacggcaacttatatgtctaaaatggaagagatttcaaggattgcctatcaagatagaaagaaaaacctaggggatccaaattacgttaatatggatccaaataaaatggtgagtgacaaatatatatcaacaatgaagaatgagaatggtgatgcgctttcggaagcagagcatgaaagcacaacgcattttgttatcattgatagagatggaacggttgtctcttcaactaatacactaagcaatttctttggaacaggaaagtacacagcagggttcttcttaaataatcaattgcagaactttggaagtgagggatttaatagttatgaacctggtaaacgttcacgaacgtttatggcccccactgtattaaagaaagatggggaaacgatcggcattgggtcaccaggtgtaaccgtattccgcaaattttaaccccaatattggataaatatacgcatggtaagggtagcttgcaagacattatcaatgaataccgttttacttttgaaaaaaatacagcgtatacagagattcagctaagttcagaagtgaaaaatgagttatctagaaaaggattgaacgtaaagaagaaagtatcccctgccttttttggtggggtacaggccttaattaaagacgagagagataatgttatcaccggcgctggagatggcagaagaaatggaacttggaaatcaaataaatag

SEQ ID NO: 2 - protein, 501 aa, B. Anthracis
SFNKIKDSVKQKIDSMGDKGTYGVSASHPLAVEEGMKVLKNGGSAVDAAIVVSYVLGVVELHASGIGGGGGMLIISKDKETFIDYRETTPYFTGNQKPHIGVPGFVAGMEYIHDNYGSLPMGELLQPAINYAEKGFKVDDSLTMRLDLAKPRIYSDKLSIFYPNGEPIETGETLIQTDLARTLKKIQKEGAKGFYEGGVARAISKTAKISLEDIKGYKVEVRKPVKGNYMGYDVYTAPPPFSGVTLLQMLKLAEKKEVYKDVDHTATYMSKMEEISRIAYQDRKKNLGDPNYVNMDPNKMVSDKYISTMKNENGDALSEAEHESTTHFVIIDRDGTVVSSTNTLSNFFGTGKYTAGFFLNNQLQNFGSEGFNSYEPGKRSRTFMAPTVLKKDGETIGIGSPGGNRIPQILTPILDKYTHGKGSLQDIINEYRFTFEKNTAYTEIQLSSEVKNELSRKGLNVKKKVSPAFFGGVQALIKDERDNVITGAGDGRRNGTWKSNK

SEQ ID NO: 3 - DNA, 627 bp, Bacteriaphage PhiNIT1
atggcacaaacagacacatatccaaatattgaagcactagagaacgcagaaactgtcggagtagcgtacaatatcgaggttaagcgccaaaatcctagtatgatctatttttcaccgcatgctggaggaattgaagtaggtactacagagcttatctaccgagtcgttgaattgaccgggggaagcctatacttgttccaagggctgctgccaagtggaaacagtcgattacacgtcacaagtacgcatttcgatgaacctatggcagtgtgtatgctttctaagcatacagacgccgtatcattccatgggtacaaggatgactacaacaagaacacgctggtgggcggactgaatacagagcttagaaacctcattgtcagcaaactcaactctaaagggattgctgctgaagtagctacagaccgctttacagctactgacccggacaacattgtaaaccgctgcgcttccggtaaaggagttcagttagagatcagctccgcacagcgtagggcttttttccagaataatgactggtctaaagccaatagagggaacgtcactcaagagttcttagactatgcagaggctattaaagaagcagaggcagagtattatggctaa

SEQ ID NO: 4 - protein, 208 aa, Bacteriaphage PhiNIT1
MAQTDTYPNIEALENAETVGVAYNIEVKRQNPSMIYFSPHAGGIEVGTTELIYRVVELTGGSLYLFQGLLPSGNSRLHVTSTHFDEPMAVCMLSKHTDAVSFHGYKDDYNKNTLVGGLNTELRNLIVSKLNSKGIAAEVATDRFTATDPDNIVNRCASGKGVQLEISSAQRRAFFQNNDWSKANRGNVTQEFLDYAEAIKEAEAEYYG

SEQ ID NO: 5 - DNA, 38 bp, Artificial sequence
  Primer
gtcgctcgagtctttcaataaaataaaagacagtgtta

SEQ ID NO: 6 - DNA, 48 bp, Artificial sequence
  Primer
gcggcgaagcttctatttatttgatttccaagttccattctctctgcc

SEQ ID NO: 7 - DNA, 45 bp, Artificial sequence
  Primer
gcggcgcatatggcacaaacagacacatatccaaatattgaagca

SEQ ID NO: 8 - DNA, 45 bp, Artificial sequence
  Primer
gcggcgggatccttagccataatactctgcctctgcttctttaat